(19) United States
(12) Plant Patent Application Publication
(10) Pub. No.: US 2004/0073980 P1
(43) Pub. Date: Apr. 15, 2004

(54) Rhododendron rootstock named 'Rhodunter 48'

(75) Inventors: Walter Preil, Ammersbek (DE); Rainer Ebbinghaus, Ahrensburg (DE)

Correspondence Address: WEBB ZIESENHEIM LOGSDON ORKIN & HANSON, P.C., 700 Koppers Building, 436 Seventh Avenue, Pittsburgh, PA, 15219-1818, US

(73) Assignee: Inkarho GmbH

(21) Appl. No.: 10/270,093

(22) Filed: Oct. 11, 2002

(51) Int. Cl. A01H 5/00

(52) U.S. Cl. PLT/238

(57) ABSTRACT

A new and distinct Rhododendron hybrid plant used as a rootstock or flowering shrub that exhibits hardy and vigorous growth.

### BOTANICAL CLASSIFICATION

Rhododendron hybrid

### VARIETAL DENOMINATION

‘Rhodunter 48’

### BACKGROUND OF THE INVENTION

The present invention comprises a new and distinct cultivar of Rhododendron hybrid used as a flowering shrub or rootstock known by the varietal name ‘Rhodunter 48’, also referred to as ‘Duftheke’. The new variety was discovered at the Institute for Ornamental Plant Breeding, Ahrensburg, Germany. The new variety is the result of a planned breeding program between unpatented Rhododendron fortunei×cv. ‘Cunningham's White’. The new variety has been trial and field tested and has been found to retain its distinctive characteristics and remain true to type through successive propagations.

### DESCRIPTION OF THE DRAWINGS

The accompanying photographic drawings illustrate the new cultivar, with the color being as nearly true as is possible with color illustrations of this type.

FIG. 1 shows a side perspective view of the new variety grown to a flowering plant; and

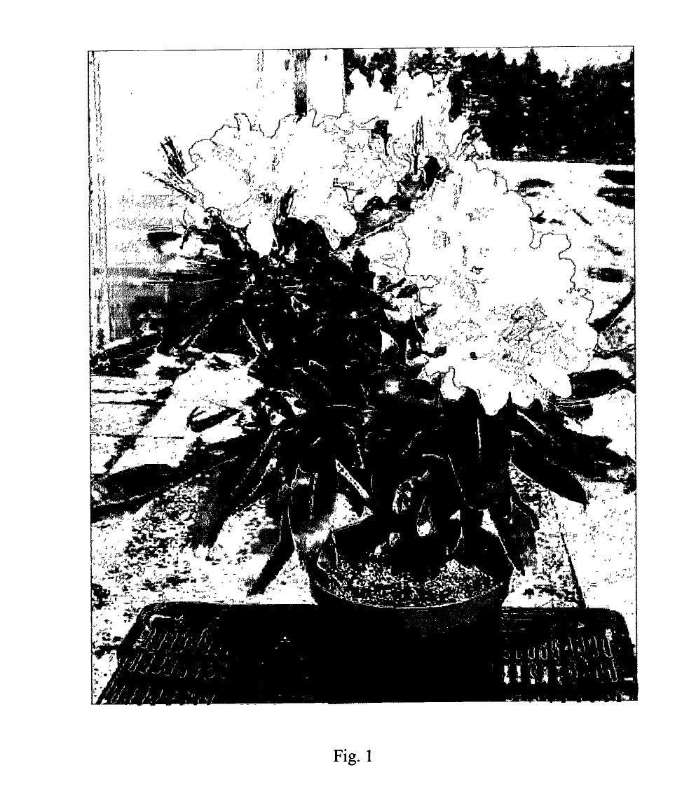

FIG. 2 shows a close-up view of the typical inflorescences of the new variety.

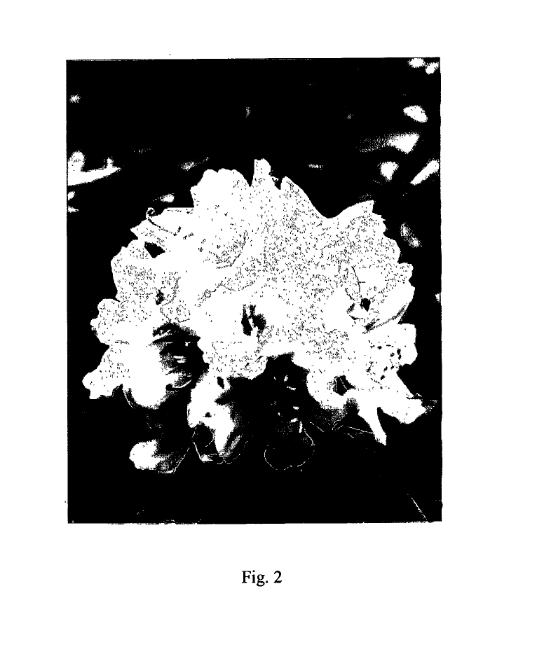

### DESCRIPTION OF THE PLANT

The following detailed description sets forth the characteristics of the new cultivar. The data which defines these characteristics were collected by asexual reproductions carried out at the Institute for Ornamental Plant Breeding in Ahrensburg, Germany. The color readings were taken in natural daylight. Color references are primarily to the R.H.S. Colour Chart of The Royal Horticultural Society of London.

### PLANT

Type: Evergreen shrub.

Use: Flowering shrub or rootstock.

Soil: Sandyloam.

Light: Full.

Fruit bearing: Very low.

Crop time: 1.5 years is required to achieve finished product size that is ready for use as a rootstock or 4 years for use as a flowering shrub.

Dimensions at crop time (3 years after grafting. Dimensions depend on the vigor of the scion cultivar):

Height.—50 cm.

Width.—40 cm.

Vigor: 120% of standard using ‘Nova Zembla’ as the standard.

Habit: Erect.

Hardiness: USDA zone 6a.

Rooting habit: Compact root bales.

Time to initiate roots: 3 months at 22-25° C. for softwood cuttings.

Disease and pest resistance: None.

Trunk (at 4 years):

Diameter.—3 cm.

Height.—5 cm above soil.

Bark color.—Surface: Grey-brown. Flesh: Grey-brown.

Lenticels.—Inconspicous.

Branches:

Color.—Surface: Grey-brown. Flesh: Grey-brown.

Internode length.—1-2 cm.

Branching angle at emergence.—45° angle.

Habit.—Compact.

Pubescence.—Absent.

Branch lenticels.—Inconspicous.

Leaves:

Arrangement.—Alternate.

Length.—20 cm maximum.

Width.—7.5 cm maximum.

Shape.—Oblanceolate.

Apex.—Broad.

Base.—Tapering.

Color.—Adaxial: 147A. Abaxial: 147C.

Surface.—Adaxial: Glabrous. Abaxial: Glabrous.

Margins.—Entire.

Division.—Undivided.

Petiole.—Length: 2 cm. Width: 3-4 mm. Color: Between 144A and 166B. Surface: Glabrous.

Leaf stipules.—Sessile.

Leaf pubescence.—Absent.

Venation pattern.—Feather-veined.

Vein color.—Adaxial: 144A. Abaxial: 145C.

Leaf texture.—Hard.

Leaf appearance.—Typical Rhododendron leaf.

Fruit: Fruits (capsules) are usually absent due to self-sterility or incompability with other Rhododendrons. When present, the seed color is 164B.

Flower:

Arrangement.—Truss.

Shape.—Widely funnel-shaped.

Bud.—Width: 20 mm. Length: 40 mm. Color: Closed: 186B. Before bursting: 65B. Time of bloom: At 2 years old. Lastingness of bloom: 21 days.

Diameter.—8 cm.

Depth.—20 mm.

Bloom quantity.—11-15 per truss.

Number of petals.—7.

Petals.—Fused.

Petal margin.—Undulate.

Flower color when fully opened.—Upper surface: 158B. Lower surface: 159C.

Peduncle.—Length: 4 cm. Width: 2 mm. Surface: Glabrous. Color: Upper surface: 179B. Lower surface: 144C.

Natural flowering season: Beginning of May through the end of May.

Fragrance: Rhododendron fortunei-like.

### REPRODUCTIVE ORGANS

Stamens:

Number.—14.

Length.—30 mm.

Color.—150D.

Anthers:

Shape.—Normal Rhododendron type.

Color.—161D.

Length.—25 mm.

Width.—1 mm.

Amount of pollen.—Medium.

Color of pollen.—150D.

Stigma color.—145A.

Pistils:

Length.—5 cm.

Style:

Color.—150C.

Form.—Filiform.

Length.—45 mm.

Width.—1 mm.

Ovaries:

Length.—5 mm.

Width.—3 mm.

Color.—137C.

Position.—Superior.

I claim:

1. A new and distinct variety of Rhododendron hybrid plant used as rootstock or flowering shrub substantially as shown and described.
